(19) United States
(12) Patent Application Publication
(10) Pub. No.: US 2022/0175625 A1
(43) Pub. Date: Jun. 9, 2022

(54) NEW COSMETIC USE OF POROUS SPHERES OF METAL OXIDE

(71) Applicant: BASF Beauty Care Solutions France S.A.S., Lyon (FR)

(72) Inventors: David HERAULT, Fericy (FR); Christine JEANMAIRE, Essey-les-Nancy (FR); Corinne THIEL, Guenange (FR)

(21) Appl. No.: 17/437,994

(22) PCT Filed: Mar. 11, 2020
(86) PCT No.: PCT/FR2020/050504

§ 371 Date: Sep. 10, 2021

(30) Foreign Application Priority Data
Mar. 12, 2019 (FR) 1902512

(51) Int. Cl. A61K 8/02; A61K 8/25; A61K 8/29; A61Q 19/00; A61Q 5/00

(52) U.S. Cl. CPC A61K 8/025; A61K 8/0279; A61K 8/25; A61K 2800/651; A61Q 19/00; A61Q 5/00; A61K 2800/412; A61K 8/29

(57) ABSTRACT

The present invention relates to the cosmetic use of porous spheres comprising a metal oxide for improving the appearance and/or comfort of the skin, skin integuments, notably the hair, and/or mucous membranes. The invention also relates to a cosmetic or pharmaceutical composition comprising said spheres.

The present invention relates to the cosmetic use of porous metal oxide spheres for improving the aesthetic appearance and comfort of the skin, skin integuments, in particular the hair, and/or mucous membranes, and also to said cosmetic compositions and to the cosmetic treatment processes resulting therefrom. The present invention also relates to pharmaceutical compositions, notably dermatological compositions, comprising porous metal oxide spheres, for improving the comfort of the skin, skin integuments and/or mucous membranes presenting a pathological state.

Visibly improving the appearance of the skin and the hair is one of the main challenges of cosmetics. Cosmetic product consumers are constantly in search of cosmetic solutions for effectively masking the signs on the skin and/or the hair that are considered to be unaesthetic. These signs appear naturally with ageing, but may also result from chronic exposure to various factors. They may be environmental factors such as pollution, the wind, climatic variations, notably variations in excessive temperatures in particular due to air conditioning, and dryness. They may also be aggressive factors of mechanical nature such as shaving, friction and hair removal, and/or aggressive factors of chemical nature such as hair treatments, notably bleaching, dyeing, permanent waving and relaxing, detergents and exfoliant care treatments. These factors cause damage to the skin, skin integuments and/or mucous membranes which are reflected by visible manifestations that are often considered unaesthetic. Intrinsic factors contribute towards their appearance, notably stress, hormone variations, weight gain, dieting and dehydration. Genetics, notably the skin type (sensitive, hyperseborrhoeic, reactive, etc.) and the ethnic origin, for example Caucasian, Asiatic or African, also play an important role in the appearance of certain manifestations.

The unaesthetic skin manifestations are notably wrinkles and fine lines, for instance the lion wrinkle and crow's feet. These wrinkles are first superficial and then become deeper, for instance the nasolabial folds. The skin manifestations also comprise the signs of loss of volume, notably collapse of the skin, slackening of the contours of the face leading to collapse of the lines of the face, giving a sad or tired appearance, or else the loss of density which is reflected by thinner, embrittled skin, accompanied by a loss of radiance and/or a dull complexion. Thus, the unaesthetic skin manifestations also include pigmentary manifestations such as the loss of homogeneity of the complexion, loss of radiance, dark rings or bags under the eyes, rashes, liver spots or age spots or white spots, notably vitiligo, rosacea, a pregnancy mask, melasma, naevus and angioma. They also include skin imperfections such as blackheads, visible pores, spots, scars, notably acne marks, burns, injuries and stretchmarks, but also rough skin and desquamation. On the mucous membranes, the unaesthetic manifestations are generally dehydration marks such as squamae and/or cracks. On the hair, the unaesthetic manifestations are generally dull, brittle hair, which is difficult to style, notably unmanageable, with split ends.

These manifestations, particularly those which appear on the face and around the eyes, are particularly perceived as unaesthetic by consumers. While some of them can be corrected by cosmetic surgery, this is not always the case and the result is occasionally unsatisfactory since it is partial, perceived as unnatural, irritating or even painful and often expensive. Many consumers do not have the means to afford such treatments or are not in favour of them. Many cosmetic alternatives exist for masking or reducing these undesirable manifestations. Makeups, in particular foundations, are aimed at providing a solution for hiding wrinkles and unifying the complexion. However, consumers are also in search of a healthy complexion effect which remains natural, without any transfer of pigments onto clothing, and also a well-being effect during application.

Patent application WO 2016/198537 describes porous particles of inorganic material containing spherical pores and covered with a hydrophobic coating.

These particles are intended to lighten or modify the colour of human keratin material and to homogenize the colour of the complexion by an immediate effect.

However, these particles are not spheres but have a polydisperse form as indicated on page 10, line 16 of said document. In addition, they are of irregular morphology. Furthermore, the presence of a hydrophobic coating is obligatory. Now, the size of the particles has an impact on the light scattering and thus on the effect of the particles on the skin. The fact that the particles of said document do not have a regular and in particular monodisperse size is thus a drawback since this does not make it possible to precisely choose the optical effect that the particles will have on the skin and undoubtedly explains the obligatory presence of a hydrophobic coating which may have consequences on the skin such as allergy and intolerance. There is thus a need to find a better alternative to these particles.

The present invention is thus directed towards meeting this ongoing need for a cosmetic alternative for improving the appearance of the skin, skin integuments and/or mucous membranes, in particular by masking the unaesthetic manifestations. The present invention is also directed towards providing a novel cosmetic ingredient which is also easy to formulate and free of side effects, and notably which does not give rise to any skin irritation.

The Applicant has discovered that metal oxide spheres have the property of improving the appearance and/or comfort of the skin and/or mucous membranes and/or skin integuments, in particular the hair.

Specifically, such spheres have the unique capacity of reflecting and scattering light and thus make it possible to smooth out the surface of the skin, skin integuments and/or mucous membranes and thus hide their unaesthetic manifestations and enhance their radiance. They also give the composition containing them a soft, silky feel.

The spheres according to the invention offer the advantage of being suitable for any skin irrespective of the type, the flesh tone or the radiance. Another advantage of the present invention is that the spheres have great stability and may be used in powder or composition form. They are inert, which makes them easy to formulate in any nature of cosmetic or dermatological compositions, whether such compositions are lipophilic or hydrophilic.

Another advantage is that the spheres may be used with the conventional ingredients of cosmetic compositions, notably pigments or colouring agents, to contribute towards masking the unaesthetic manifestations, in particular imperfections.

According to an advantageous embodiment, the spheres according to the invention may be used in foundations and/or makeup products.

One subject of the present invention is thus the cosmetic use of porous spheres comprising a metal oxide for improving the appearance and/or comfort of the skin, skin integuments, notably the hair, and/or mucous membranes.

According to a preferential embodiment, the porous spheres have a mean diameter ranging from 0.5 μm to 100 μm, advantageously from 1 μm to 100 μm, in particular from 4.5 μm to 9.9 μm, and/or a mean porosity ranging from 0.10 to 0.90, preferentially from 0.10 to 0.80 and/or a mean pore diameter ranging from 50 nm to 999 nm, advantageously from 50 nm to 500 nm, in particular from 100 nm to 400 nm, more particularly from 220 nm to 300 nm.

Advantageously, the porous spheres have a mean diameter ranging from 0.5 μm to 100 μm, advantageously from 1 μm to 100 μm, in particular from 4.5 μm to 9.9 μm, and a mean porosity ranging from 0.10 to 0.90, preferentially from 0.10 to 0.80 and a mean pore diameter ranging from 50 nm to 999 nm, advantageously from 50 nm to 500 nm, in particular from 100 nm to 400 nm, more particularly from 220 nm to 300 nm.

Preferentially, the metal oxide of the spheres is chosen from the oxide silica, titania, alumina or zirconia, cerium oxide, iron oxide, zinc oxide, indium oxide, tin oxide, chromium oxide, mixed metal oxide and combinations thereof, preferentially silica, titania, alumina and zinc oxide and combinations thereof, more preferentially silica and titanium oxides and combinations thereof.

Preferentially, the porous spheres comprise from 60% by weight to 99.9% by weight of metal oxide and from 0.1% by weight to 40% by weight of light-absorbing agents, relative to the total weight of the spheres.

Preferentially, the spheres have a mean diameter ranging from 1 μm to 75 μm and a mean porosity ranging from 0.45 to 0.65.

According to a particular embodiment of the invention, the spheres have a mean diameter ranging from 4.5 μm to 9.9 μm, a mean porosity ranging from 0.45 to 0.65 and a mean pore diameter ranging from 220 nm to 300 nm.

A subject of the present invention is also cosmetic compositions containing these porous spheres, in particular for the uses mentioned.

A subject of the present invention is also a pharmaceutical composition, preferentially a dermatological composition, which is useful for improving the comfort of the skin, integuments and/or mucous membranes, in particular having a pathological state.

Such cosmetic or pharmaceutical compositions, preferentially dermatological compositions, comprise the spheres in a concentration ranging from 1×10⁻³ to 100% by weight, preferentially from 0.5% to 5% by weight relative to the total weight of the composition.

The use according to the invention is preferentially cosmetic, and by topical application to at least one concerned area or part of healthy skin, healthy mucous membrane and/or healthy skin integuments, preferentially the hair, preferentially in humans.

For the purposes of the present invention, the term “cosmetic use” means a non-therapeutic, non-pharmaceutical use of the spheres according to the invention, preferentially on healthy skin, notably a healthy scalp, and/or healthy skin integuments, in particular healthy hair, and/or healthy mucous membranes.

For the purposes of the present invention, the expression “part of the skin, preferentially of the scalp, and/or area of skin, preferentially of the scalp, referred to as “healthy”” means a part of the skin, preferentially of the scalp, and/or an area of skin, preferentially of the scalp, referred to as non-pathological by a dermatologist, that is to say which does not present any infection, inflammation, disease or ailment of the skin, such as folliculitis, candidiasis, psoriasis, ichthyosis, pathological conditions, wounds or injuries.

The term “topical application”, used herein, means applying the spheres according to the present invention optionally in the form of an active ingredient and/or of a composition to the surface of the skin, including the scalp, skin integuments, notably the hair, and/or mucous membranes, notably by direct application or by spraying.

The term “improving the appearance” means decreasing or reducing the visibility of the unaesthetic manifestations, in particular masking or hiding them, preferentially those present on the face, notably by filling and/or by an optical effect.

According to the present invention, the “unaesthetic manifestations” are unaesthetic irregularities in the relief and/or texture of the skin, mucous membranes and/or skin integuments and/or irregularities in the colour of the skin, mucous membranes and/or skin integuments.

According to the present invention, the unaesthetic irregularities in the relief and texture of the skin are wrinkles and fine lines, notably the lion wrinkle and crow's feet, raised points, blackheads, dilated pores, a rough appearance, marks and scars, notably those caused by injuries, acne, spots, burns and/or stretchmarks.

According to the present invention, the irregularities in the colour of the skin are pigmentation marks, in particular white or dark spots, notably age marks or hyperpigmented or depigmented marks, a pregnancy mask, beauty spots, melanomas, freckles and rashes, but also a loss of radiance of the complexion, a dull complexion, dark rings and bags under the eyes, rosacea and couperosis.

According to a preferential embodiment, the unaesthetic manifestations are selected from wrinkles, fine lines, blackheads, visible pores, a loss of radiance of the complexion, a dull complexion, dark rings and bags under the eyes, pigmentation marks and any mixture thereof.

The unaesthetic manifestations of the skin integuments, preferentially the hair, are the dull, brittle appearance. For the bodily hairs and head hair, the manifestations also include the brittle, friable, damaged appearance and/or difficulty in shaping them, notably for the hair, and in styling them, and/or split ends.

The unaesthetic manifestations of the mucous membranes are notably the dull, cracked, wizened and/or damaged appearance and also squamae.

In general, the improvement of the unaesthetic manifestations may be visualized and evaluated by microscopy and/or by analysis of high-resolution photography notably with measurement of the gloss of the area of skin, mucous membrane and/or skin integuments.

The improvement of the radiance or glow of the complexion may notably be measured by means of an objective instrumental method. This in vivo measurement method consists in taking high-resolution photographs in cross-polarized configuration of volunteers' faces taken at 45° before and after application of the test product. On the basis of these digital photographs, image analysis enables the extraction and quantifying of specific parameters (for example: L*, a*, b*, C, h°) related to the colour, the radiance, the homogeneity, and the texture of the skin.

Similarly, the gloss may notably be measured according to this method on the basis of high-resolution photographs in cross-polarized and parallel-polarized configuration of volunteers' faces taken at 45° before and after application of the test product. On the basis of these digital photographs, image analysis enables the extraction and quantifying of specific parameters associated with the gloss such as the specular gloss and the contrast gloss. For the purposes of the present invention, the term “reducing the visibility of skin pores” means masking the skin pores notably by filling and/or by an optical effect.

The visibility of skin pores may be demonstrated in vivo by an objective instrumental method (image analysis) that makes it possible to extract and quantify specific parameters from high-resolution photographs, in cross-polarized configuration, of volunteers' faces taken before and after application of a composition comprising the spheres according to the invention. The density of the skin pores may also be measured in vivo by imaging, notably by the fringe projection technique, by measuring the parameter known as curvature.

In one preferred embodiment of the invention, the spheres according to the invention are in an effective amount for reducing skin pore visibility by at least 10%, preferentially by at least 20%, after application of a cream comprising the spheres according to the invention, more preferentially prepared under the conditions described in one of the Examples 1 to 3, preferentially formulated in the form of a cosmetic composition as described in Example 6.

The term “mucous membrane” means the ocular mucous membrane, the vaginal mucous membrane, the urogenital mucous membrane, the anal mucous membrane, the nasal mucous membrane and/or the buccal, labial and/or gingival mucous membrane, preferentially the ocular and/or buccal mucous membranes.

In the context of the present invention, the term “improving the comfort” means increasing the organoleptic properties, in particular the feel and the ease of spreading of the composition, notably by a soft, silky, light and smooth feel, but also giving a sensation of suppleness to the area to which the composition containing the spheres has been applied. The organoleptic properties and notably the feel of a composition may be evaluated according to the conventional methods in the field, notably by evaluation by individuals trained to perform sensory tests on the skin. For better evaluation, the measurement is performed by comparison with a placebo, i.e. the same composition which does not contain the spheres according to the invention. This evaluation is preferentially performed on an emulsion.

Advantageously, the improvement of the comfort is analysed by sensory evaluation of a cream as described in Example 6. A panel of trained individuals evaluates the compositions by applying a given amount of composition to their forearm. According to a defined procedure, the composition is spread on the skin and its specific characteristics are evaluated, for instance its absorption, the softness and the appearance in comparison with a placebo composition. A value from −1 to +1 is given. For example, for the lightness sensation, the test composition may be evaluated as +1 to indicate that it is much lighter than the “placebo” reference composition or slightly less light than the placebo composition with a value of −0.5.

If two creams afford the same perception, then the individuals indicate a value of 0 for the parameter evaluated. Such evaluations are made in the same manner for the test composition and the reference composition, under the same climatic conditions and under blind conditions. A statistical analysis then makes it possible to evaluate the significance and the deviations in the evaluation results.

The term “skin integuments” means the hair, the eyelashes, the eyebrows, the beard, the moustache and/or the nails, preferentially the hair.

Advantageously, the use according to the present invention makes it is possible to improve the appearance of the skin by improving the radiance of the complexion, making it more uniform and shiny, affording a fresh, luminous, healthy complexion effect, in particular by hiding the unaesthetic texture, relief and/or colour manifestations of the skin.

According to another embodiment, the spheres also make it possible to soften skin which shows signs of discomfort, notably skin which has been exposed to aggressive conditions of mechanical nature such as shaving, friction and hair removal, and/or of chemical nature such as hair treatments, notably bleaching, dyeing, permanent waving and relaxing, detergents and exfoliant care treatments.

In a particular embodiment, the use according to the present invention makes it possible to lighten and eliminate the skin, skin integuments, notably the hair, and/or the mucous membranes, to make them more uniform, to create a soft-focus effect, to soften the lines, to fill in irregular lines and marks notably as a filling agent, in particular wrinkles.

The spheres according to the invention are porous metal oxide spheres. Such spheres are photon beads, which means that they have a degree of periodic colour variations.

The spheres according to the invention comprise a metal oxide and preferentially have a mean diameter ranging from about 0.5 μm to about 100 μm, advantageously from 1 μm to 100 μm, in particular from 4.5 μm to 9.9 μm, and/or a mean porosity ranging from about 0.10 to about 0.90 or from about 0.10 to about 0.80 and/or a mean pore diameter ranging from about 50 nm to about 999 nm, advantageously from 50 nm to 500 nm, in particular from 100 nm to 400 nm, more particularly from 220 nm to 300 nm.

The porous spheres comprising a metal oxide may be prepared using a sacrificial polymer model. Such porous spheres show a colour that is observable to the human eye.

According to one embodiment, an aqueous colloidal dispersion containing polymer particles and a metal oxide is prepared, the polymer particles typically being nanometre-sized. The aqueous colloidal dispersion is then mixed in an oily continuous phase, for example with a microfluidic device, to produce a water-in-oil emulsion. Aqueous droplets of emulsion are obtained, collected and dried to form the spheres containing the nanoparticles of polymer and of metal oxide. The nanoparticles (nanospheres) are then removed, for example by calcining, to give micrometre-sized spherical particles of metal oxide containing a high degree of porosity and nanometre-sized pores. The spheres may contain uniform pore diameters, due to the use of spherical and monodisperse polymer particles.

A drop of emulsion containing polymer nanospheres and a metal oxide is dried to remove the solvent, thus producing a sphere formed from polymer nanospheres with the metal oxide in the interstitial spaces between the polymer nanospheres (model microspheres or “direct structure”).

The polymer nanospheres define an interstitial space. Calcining results in removal of the polymer, thus providing a metal oxide sphere which has high porosity or a large interstitial volume (inverse structure). The porous metal oxide spheres are advantageously sintered, leading to a thermally and mechanically stable, consolidated, continuous solid structure.

In certain embodiments, the formation and collection of the droplets are performed in the microfluidic device.

Microfluidic devices are, for example, devices with a narrow channel having a microscopic-sized junction suitable for producing uniform droplets, connected to a recovery tank.

Microfluidic devices contain, for example, a junction having a channel width ranging from 10 μm to about 100 μm. These devices are made, for example, from polydimethylsiloxanes (PDMS) and may be prepared, for example, by soft lithography. An emulsion may be prepared inside the device by pumping an aqueous dispersed phase and an oily continuous phase in a given ratio, thus forming an emulsion by mixing the two phases. Alternatively, an oil-in-water emulsion may be used.

According to the invention, the metal oxides include oxides of transition metals, metalloids and earth metals that are compatible with cosmetic and/or dermatological use, for instance the oxide silica, titania, alumina or zirconia, cerium oxide, iron oxide, zinc oxide, indium oxide, tin oxide, chromium oxide, mixed metal oxide, and combinations thereof, preferentially chosen from silica and titanium oxides and/or combinations thereof.

The polymer nanoparticles/metal oxide weight ratio (weight/weight) ranges, for example, from about 0.1/1 to about 10.0/1 or from about 0.5/1 to about 10.0/1. Advantageously, this ratio is: 0.1/1, 0.5/1, 1.0/1, 1.5/1, 2.0/1, 2.5/1, 3.0/1, 3.5/1, 4.0/1, 5.0/1, 5.5/1, 6.0/1, 6.5/1, 7.0/1, 8.0/1, 9.0/1 or 10.0/1.

The continuous oily phase comprises, for example, an organic solvent, a silicone oil or a fluoro oil. According to the invention, the term “oil” denotes a water-immiscible organic phase. The organic solvents include hydrocarbons, such as heptane, hexane, toluene and xylene, and also alcohols, such as methanol, ethanol, propanol, etc.

The emulsion droplets are collected and dried and the polymer is removed. Drying is performed according to the conventional techniques, for example by microwave irradiation, in a thermal oven, under vacuum, in the presence of a dehydrating agent/absorbent or a mixture of these techniques.

The polymer is removed according to the conventional techniques, for example by calcining, pyrolysis or with a solvent. The calcining is performed according to one embodiment at temperatures ranging from 200° C. to about 1200° C. According to one embodiment, the calcining temperature is at least 200° C., preferentially at least 500° C., more preferentially at least 1000° C. Alternatively, the calcining temperature is from 200° C. to about 700° C. The calcining is performed for a suitable time ranging, for example, from 0.1 hour to about 12 hours, preferentially from 1 hour to 8 hours. In certain embodiments, the calcining is performed for at least 0.1 hour, for at least 1 hour, for at least 5 hours or for at least 10 hours.

Alternatively, the liquid dispersion comprising the nanoparticles of polymer and of metal oxide is formed in oily dispersed phase and in continuous aqueous phase to form an oil-in-water emulsion. The oily droplets may thus be collected and dried in the same way as the aqueous droplets.

The spheres are spherical or virtually spherical and are micrometre-sized. They thus have a mean diameter ranging from 0.5 micron (μm) to about 100 μm, advantageously from 1 μm to 100 μm, in particular from 4.5 μm to 9.9 μm. The polymer nanoparticles employed as model are also spherical, at the nanometric scale, they are monodisperse and have a mean diameter ranging from 50 nm to about 999 nm, advantageously from 50 nm to 500 nm, in particular from 100 nm to 400 nm. The metal oxide used may also be in the form of particles, such particles then possibly being nanometre-sized.

The metal oxide in dispersion may be provided in the form of metal oxide or in the form of a metal oxide precursor, for instance via the sol-gel technique.

Drying of the polymer/metal oxide droplets followed by removal of the polymer makes it possible to obtain spheres containing uniform cavities (pores). In general, in the processes described, each droplet forms a single microsphere. The pore diameters are dependent on the size of the polymer particles. Shrinkage or compaction may appear during the removal of the polymer, producing pore sizes that are slightly smaller than the initial size of the polymer particles, for example ranging from 10% to 40% smaller than the size of the initial polymer particle. The pore diameter is just as uniform as were the shape and size of the polymer particles.

The pore diameter of the spheres according to the present invention ranges from about 50 nm to about 999 nm, advantageously from 50 nm to 500 nm, in particular from 100 nm to 400 nm.

The mean porosity of the spheres may be relatively high, for instance from about 0.10 or about 0.30 to about 0.80 or about 0.90. The mean porosity of a microsphere denotes the total volume of the pores, as a fraction of the volume of the entire microsphere. The mean porosity may also be referred to as the “volume fraction”. This is a dimensionless magnitude.

In certain embodiments, the porous sphere has a solid centre or core and porosity towards the outer surface of the sphere.

In other embodiments, the porous spheres have an empty centre with a porosity which increases towards the interior of the spheres.

In certain embodiments, there is thus a porosity gradient towards the centre or towards the exterior of the sphere.

In other preferential embodiments of the invention, the porosity is uniformly distributed throughout the volume of the spheres.

In the porous sphere according to the invention, the mean diameter of the spheres is markedly larger than the mean diameter of the pores. For example, the mean diameter of the spheres is generally at least 25 times, preferentially at least 30 times, preferentially at least 35 times, more preferentially 4 times greater than the mean diameter of the pores.

In certain embodiments, the ratio of the mean diameter of the microspheres to the mean diameter of the pores is at least 40/1, at least 50/1, at least 60/1, at least 70/1, at least 80/1, at least 90/1, at least 100/1, at least 110/1, at least 120/1, at least 130/1, at least 140/1, at least 150/1, at least 160/1, at least 170/1, at least 180/1, at least 190/1, at least 200/1, at least 210/1, at least 220/1, at least 230/1, at least 240/1, at least 250/1, at least 260/1, at least 270/1, at least 280/1, at least 290/1, at least 300/1, at least 310/1, at least 320/1, at least 330/1, at least 340/1 or at least 350/1.

Models of polymer spheres comprising monodisperse polymer nanospheres may result in metal oxide spheres having pores of similar diameter, once the polymer is removed.

Without being bound to a theory, it is thought that the majority of the spheres have a saturated colour with reduced light scattering when the sphere porosity and/or diameter and/or the pore diameter are within certain ranges of values. These colour properties are particularly important and can be adjusted according to the desired intensity of light scattering on the skin, the skin integuments and/or the mucous membranes. In certain embodiments, it is preferable to have white spheres. The spheres according to the invention make it possible to obtain smaller light scattering and greater reflection (haze) of the light at lower doses than the particles of the prior art, all the more so in the mean diameter range of the spheres ranging from 4.5 μm to 9.9 μm and/or in the mean diameter range of pores ranging from 50 nm to 500 nm, in particular from 100 nm to 400 nm, more particularly from 220 nm to 300 nm.

The porous spheres according to the invention mainly contain the metal oxide. Advantageously, they consist essentially of the metal oxide. Preferentially, they consist exclusively of metal oxide.

Preferentially, the majority of the porous spheres have a colour that is observable by the naked eye. One or more light-absorbing agents may also be present in the spheres, thus making it possible to provide a more saturated observable colour. Such absorbing agents are added, for example, during the physical mixing of the spheres or may be included in the droplets before drying. Thus, the spheres according to the invention do not have any observable colour without a light-absorbing agent and have an observable colour when a light-absorbing agent is added.

The spheres then have the advantage of being able to be used as colouring agent in cosmetic and/or dermatological compositions.

The porous spheres according to the present invention may have a colour which is or is not angle-dependent. The term “angle-dependent colour” means an observed colour which depends on the angle of the incident light or on the angle between the observer and the observed area of colour. An “angle-independent colour” means that the observed colour does not substantively depend on the angle of the incident light or on the angle between the observer and the observed area of colour.

Spheres having an angle-dependent colour may notably be obtained by using monodisperse polymer nanospheres. They may also be obtained when the step of drying of the liquid droplets for providing the polymer sphere model is performed gently, thus allowing the polymer nanospheres to become ordered.

Spheres whose colour is angle-independent may be obtained when the step of drying of the liquid droplets is performed rapidly, not allowing the polymer nanospheres to become ordered.

The porous spheres may thus comprise, for example, from 60% by weight to 99.9% by weight of metal oxide and from 0.1% by weight to 40% by weight of light-absorbing agents, relative to the total weight of the spheres. According to one embodiment of the invention, the spheres contain 0.3%, preferentially 0.5%, preferentially at least 1% and more preferentially at least 5% by weight of light-absorbing agents relative to the total weight the spheres.

According to an alternative and preferential embodiment, the spheres do not contain any light absorber and the cosmetic composition which contains them does not contain any light absorber either. Specifically, light absorbers such as TiO₂ may have a certain toxicity. Now, the inventors have discovered, surprisingly, that even without light absorber, the spheres according to the invention have an immediate radiance and imperfection-masking effect on the skin or anti-blue light filter booster effect by virtue of their power for scattering daylight and/or blue light.

Advantageously, the light-absorbing agent(s) are selected from the group consisting of organic and inorganic pigments, which are compatible with cosmetic and/or dermatological application, in particular to the skin, mucous membranes and/or skin integuments.

Advantageously, the porous spheres are themselves also monodisperse.

According to the invention, the particle size is synonymous with the particle diameter and is determined, for example, by scanning electron microscopy (SEM) or transmission electron microscopy (TEM). The term “mean particle size” (or mean diameter) means the D50, meaning that half the population is below this size and the other half is above it. The particle size is that of the primary particles and may be measured by laser light scattering techniques, on dispersions or dry powders.

An analysis by mercury porosimetry is used to characterize the porosity of the spheres. Mercury porosimetry consists in applying a controlled pressure in the sample to be measured immersed in mercury. The external pressure is applied so that the mercury penetrates into the pores/holes of the material. The amount of pressure required is inversely proportional to the size of the pores/holes. Mercury porosimetry thus gives a volume and size distribution of the pores. Mercury porosimetry generates volume and pore size distributions from the pressure data relative to the intrusion data generated by the instrument, by means of the Washburn equation. Thus, for example, the porous silica spheres contain pores with a mean diameter of 165 nm and have a mean porosity of 0.8.

The term “the majority of the spheres” means the population of the spheres. It may be an amount ≥≥0.1 mg, ≥0.2 mg, ≥0.3 mg, ≥0.4 mg, ≥0.5 mg, ≥0.7 mg, ≥1.0 mg, ≥2.5 mg, ≥5.0 mg, ≥10.0 mg or ≥25.0 mg. The majority of the spheres may be free of other compounds.

The term “have a colour that is observable with the naked eye” means a colour that is observable by a majority of people. This may be for any sample of spheres distributed on a given surface, for example on 1 cm², 2 cm², 3 cm², 4 cm², 5 cm², 6 cm², 7 cm², 8 cm², 9 cm², 10 cm², 11 cm², 12 cm², 13 cm², 14 cm² or 15 cm². This may also mean that it is observable by the CIE 1931 2° observation standard and/or by the CIE 1964 10° observation standard. The colour observation background may be of a different kind, for example white, black or any intermediate between black and white.

The term “micrometre-sized” or “micro-” means a size ranging from about 1 μm to about 999 μm. The term “nanometre-sized” or “nano-” means a size ranging from about 1 nm to about 999 nm.

The term “monodisperse” refers to the population of porous spheres or of nanospheres of polymer particles which have a homogeneous shape and a homogeneous diameter. The population of spheres is thus said to be monodisperse when 90%, 91%, 92%, 93%, 94%, 95%, 96%, 97%, 98% or 99% of the number of particles have a diameter of ±7%, ±6%, ±5%, ±4%, ±3%, ±2% or ±1% relative to the mean diameter of the population of spheres.

Removal of the monodisperse population of polymer nanospheres gives metal oxide spheres with a population of pores having a mean diameter.

Thus, the spheres according to the invention comprising a metal oxide are prepared via a method comprising the following steps:
forming a liquid dispersion of nanoparticles of polymer and of a metal oxide;
forming liquid droplets of the dispersion;
drying the liquid droplets of the spheres comprising the polymer nanospheres and the metal oxide;
removing the polymer nanospheres from the model spheres to provide the porous metal oxide spheres.

According to an advantageous embodiment, the liquid droplets are aqueous.

According to an alternative, the liquid droplets are oily.

According to a preferential embodiment, the method comprises a continuous phase and mixing of the liquid dispersion with this continuous phase to form an emulsion containing the droplets of dispersed liquid dispersion. According to a particular embodiment, the continuous phase is oily and mixing is performed between the continuous oily phase and the aqueous liquid dispersion to form a water-in-oil emulsion containing aqueous droplets.

According to an alternative embodiment, the continuous phase is aqueous and mixing is performed between the continuous aqueous phase and the oily liquid dispersion to form an oil-in-water emulsion containing lipid droplets.

According to an advantageous embodiment, the method comprises the subsequent steps of collecting the droplets, drying the droplets and removal of the polymer nanospheres from the sphere model.

Advantageously, the polymer nanoparticles have a mean diameter of 50 nm, 75 nm, 100 nm, 130 nm, 160 nm, 190 nm, 210 nm, 240 nm, 270 nm, 300 nm, 330 nm, 360 nm, 390 nm, 410 nm, 440 nm, 470 nm, 500 nm, 530 nm, 560 nm, 590 nm, 620 nm, 650 nm, 680 nm, 710 nm, 740 nm, 770 nm, 800 nm, 830 nm, 860 nm, 890 nm, 910 nm, 940 nm, 970 nm or 990 nm. Preferentially, the polymer is selected from the group consisting of poly(meth)acrylic acid, poly(meth)acrylates, polystyrenes, polyacrylamides, polyethylene, holly propylene, polylactic acid, polyacrylonitrile, and also derivatives thereof and salts thereof, copolymers thereof and combinations thereof. Preferentially, the polymer is chosen from the group consisting of polystyrenes, for example a polystyrene/acrylic acid, polystyrene/poly(ethylene glycol) methacrylate or polystyrene/styrene sulfonate copolymer.

According to a preferential embodiment, the metal oxide is chosen from the oxide silica, titania, alumina or zirconia, cerium oxide, iron oxide, zinc oxide, indium oxide, tin oxide, chromium oxide, mixed metal oxide and combinations thereof. Preferentially, the metal oxide is a silica, titania or alumina oxide or a combination thereof.

Advantageously, the porous spheres comprise between 60% and 99.9% of metal oxide, preferentially at least 70%, more preferentially at least 80%, more preferentially at least 90%, more preferentially 99.9% by weight relative to the total weight of the spheres.

According to an advantageous preferential embodiment, the porous spheres have a mean diameter ranging from 0.5 μm to about 100 μm, advantageously from 1 μm to 100 μm, in particular from 4.5 μm to 9.9 μm, a mean porosity ranging from 0.10 to 0.90, or from 0.10 to 0.80 and a mean pore diameter ranging from 50 nm to 999 nm, advantageously from 50 nm to 500 nm, in particular from 100 nm to 400 nm, more particularly from 220 nm to 300 nm.

In a preferential embodiment, the porous spheres have a mean diameter ranging from 1 μm to 75 μm, preferentially from 2 μm to 70 μm, preferentially from 3 μm to 65 μm, from 4 μm to 60 μm, from 5 μm to 55 μm or from 5 μm to 50 μm, more preferentially from 10 μm to 25 μm, in particular from 4.5 μm to 9.9 μm. In an advantageous embodiment, they are microspheres and the mean diameter thus ranges from 1 μm to 100 μm, advantageously from 1 to 20 μm, more advantageously from 3 to 20 μm, even more advantageously from 5 to 9 μm, in particular from 4.5 μm to 9.9 μm, more particularly from 4 to 8 μm. Particularly advantageously, they are not nanospheres so as to avoid penetration into the skin and to have a surface effect. The spheres according to the invention thus advantageously remain on the surface of the skin.

In a preferential embodiment of the invention, the porous spheres have a mean porosity chosen from: 0.10, 0.12, 0.14, 0.16, 0.18, 0.20, 0.22, 0.24, 0.26, 0.28, 0.30, 0.32, 0.34, about 0.36, 0.38, 0.40, 0.42, 0.44, 0.46, 0.48, 0.50, 0.52, 0.54, 0.56, 0.58, 0.60, 0.62, 0.64, 0.66, 0.68, 0.70, 0.72, 0.74, 0.76, 0.78, 0.80 or 0.90.

In an alternative embodiment of the invention, the porous spheres have a mean diameter ranging from 1 μm to 75 μm, from 2 μm to 70 μm, from 3 μm to 65 μm, from 4 μm to 60 μm, from 5 μm to 55 μm, from 5 μm to 50 μm; for example a mean diameter chosen from 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm or 25 μm.

In a preferential embodiment of the invention, the porous spheres have a mean pore diameter ranging from about 50 nm to about 999 nm, advantageously from 50 nm to 500 nm, in particular from 100 nm to 400 nm, more particularly from 220 nm to 300 nm, even more particularly chosen from: 50 nm, 60 nm, 70 nm, 80 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm or 800 nm.

In an alternative embodiment, the porous spheres according to the invention have a mean diameter chosen from 4.5 μm, 4.8 μm, 5.1 μm, 5.4 μm, 5.7 μm, 6.0 μm, 6.3 μm, 6.6 μm, 6.9 μm, 7.2 μm, 7.5 μm, 7.8 μm, 8.1 μm, 8.4 μm, 8.7 μm, 9.0 μm, 9.3 μm, 9.6 μm or 9.9 μm; and a mean porosity chosen from 0.45, 0.47, 0.49, 0.51, 0.53, 0.55, 0.57, 0.59, 0.61, about 0.63 or 0.65; and a mean pore diameter chosen from 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm or 300 nm.

According to a particular embodiment of the invention, the porous spheres have a mean diameter ranging from 4.5 μm to 9.9 μm, a mean porosity ranging from 0.45 to 0.65 and a mean pore diameter ranging from 220 nm to 300 nm.

According to a particular embodiment of the invention, the porous spheres according to the invention do not include a coating layer on their surface containing a polyorganosiloxane, in particular a silicone compound.

In an advantageous embodiment, the porous spheres according to the invention do not include a coating layer on their surface containing a hydrophobic compound. In a particularly advantageous embodiment, the porous spheres according to the invention do not include a coating layer on their surface.

According to the present invention, the porous spheres are used alone, notably in powder form (100%), or in the form of a composition, notably a cosmetic composition, at a concentration ranging from 1×10⁻³ to 100% by weight, and advantageously ranging from 1×10⁻² to 95%, preferably ranging from 0.01% to 80%, more preferentially from 0.05% to 50%, preferentially from 0.1% to 30%, preferentially up to 20%, more preferentially up to 10%, and more preferentially from 0.5% to 5% by weight relative to the total weight of the composition.

The porous spheres according to the invention may be used alone, notably in the form of a cosmetic active ingredient or in a composition intended to be in contact with the skin, skin integuments and/or mucous membranes, for instance a cosmetic composition, preferentially intended for topical application.

The term “topical application”, used herein, means applying the porous spheres according to the present invention optionally in the form of an active ingredient and/or of a composition to the surface of the skin, including the scalp, skin integuments and/or mucous membranes, notably by direct application or by spraying.

The active ingredient and/or the cosmetic compositions containing the porous spheres according to the invention are preferentially intended for the cosmetic care and/or treatment of the skin, including the scalp and also skin integuments, preferentially the hair.

In another embodiment, the spheres according to the invention may be incorporated into a cosmetic composition also comprising at least one cosmetically acceptable excipient.

For the purposes of the present invention, the term “cosmetically acceptable” excipient means a topically acceptable compound and/or solvent, that is to say one which does not induce an undue allergic response on contact with the skin, including the human scalp, and skin integuments, which is non-toxic, which is not unstable, or equivalents thereof.

The cosmetic composition according to the invention may be in any presentation form conventionally used for topical application to the skin, including the scalp, and skin integuments, such as liquid or solid forms, or even in the form of pressurized liquid. They may notably be formulated in the form of an aqueous or oily solution, notably a lotion, a cream, an aqueous gel or an oily gel, notably in a jar or a tube, notably a shower gel, a shampoo, a hair conditioner, a milk, an oil, an emulsion, a hydrogel, a microemulsion or a nanoemulsion, notably an oil-in-water or water-in-oil or multiple or silicone-based emulsion, a serum, a lotion, notably in a glass or plastic bottle or measuring bottle or an aerosol or spray, a vial, a liquid or solid soap, a paste, an ointment, a foam, a mask, a lacquer, a patch, an so anhydrous product, which is preferably liquid, pasty or solid, for example in the form of a rod, notably in stick form, or in powder form, preferentially a cream, a serum or a lotion.

It may also be a makeup product or a makeup-removing product. In particular, the cosmetic composition is chosen from the group consisting of a serum, a lotion, a cream, a shampoo, a hair conditioner, an oil, a milk, an ointment, a paste, a foam, an emulsion, a hydrogel, a shower gel, a mask, a lacquer, a spray, and a wax; it is more preferentially a cream, a serum or a lotion. It may also be a powder, notably a makeup powder, a foundation stick, compositions for caring for the beard, after-shave and/or hair-removing compositions. According to an advantageous embodiment, the cosmetic composition according to the invention is at least slightly gelled and/or comprises an oily phase, and is preferentially of oily composition type or oil-in-water or water-in-oil emulsion or lotion type.

The compositions according to the invention may contain any suitable solvent and/or any suitable vehicle and/or any suitable excipient, optionally in combination with other compounds of interest.

As a result, for these compositions, the excipient contains, for example, at least one compound chosen from the group consisting of preserving agents, emollients, emulsifiers, surfactants, moisturizers, thickeners, conditioners, mattifying agents, stabilizers, antioxidants, texturing agents, gloss agents, film-forming agents, solubilizers, pigments, colourants, fragrances and sunscreens. These excipients are preferably chosen from the group consisting of amino acids and derivatives thereof, polyglycerols, esters, cellulose polymers and derivatives, lanolin derivatives, phospholipids, lactoferrins, lactoperoxidases, sucrose-based stabilizers, vitamins E and derivatives thereof, natural and synthetic waxes, plant oils, triglycerides, unsaponifiable matter, phytosterols, plant esters, silicones and derivatives thereof, protein hydrolysates, jojoba oil and derivatives thereof, liposoluble/water-soluble esters, betaines, amine oxides, plant extracts, sucrose esters, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerol, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, glycerol, bisabolol, a dimethicone, sodium hydroxide, PEG-30 dipolyhydroxystearate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8, beeswax, glycerides from hydrogenated palm oil, glycerides from hydrogenated palm oil, lanolin oil, sesame oil, cetyl lactate, lanolin alcohol, castor oil, titanium dioxide, lactose, sucrose, low-density polyethylene, an isotonic saline solution.

Many cosmetically active ingredients are known to those skilled in the art for improving the appearance of the skin. A person skilled in the art knows how to formulate cosmetic or dermatological compositions to obtain the best effects. Moreover, the compounds described in the present invention may have a synergistic effect when they are combined with each other. These combinations are also covered by the present invention. The CTFA Cosmetic Ingredient Handbook, Second Edition (1992) describes different cosmetic and pharmaceutical ingredients commonly used in the cosmetics and pharmaceutical industry, which are suitable in particular for topical use. Examples of these classes of ingredients comprise, without being limited thereto, the following compounds: abrasives, absorbents, compounds for aesthetic purposes such as fragrances, pigments, colourants, essential oils, astringents, antiacne agents, anti-flocculant agents, antifoam agents, antimicrobial agents (for example: iodopropyl butyl carbamate), antioxidants, binders, biological additives, buffer agents, swelling agents, chelating agents, additives, biocidal agents, denaturing agents, thickeners, and vitamins, and derivatives or equivalents thereof, film-forming materials, polymers, opacifiers, pH regulators, reducing agents, depigmenting or lightening agents (for example: hydroquinone, kojic acid, ascorbic acid, magnesium ascorbyl phosphate, ascorbyl glucosamine), conditioning agents (for example: humectants).

Particularly advantageously, the spheres according to the invention may be used, optionally in a cosmetic or pharmaceutical composition, preferentially a dermatological composition, as sole agent for improving the appearance, in particular the unaesthetic manifestations and/or the comfort of the skin, skin integuments and/or mucous membranes, or in combination with other active agents having the same properties or complementary properties and which are conventional in cosmetic or dermatological compositions, for instance those chosen from:
moisturizing agents: one or more moisturization-promoting agents such as a polysaccharide extracted from Cassia angustifolia seeds sold under the name Hyalurosmooth™ by the Applicant, or an agent chosen from combinations containing pullulan, sodium hyaluronate and sodium alginate sold under the name PatcH2O™ by the Applicant or one or more compounds of Natural Moisturizing Factor or a natural extract of honey sold by the Applicant under the name Melhydran™ and/or a compound of the glucosyl glyceride family, in particular hexosyl glyceride, an extract of Litchi chinensis pericarp under the name Litchiderm™ by the Applicant;
an agent for stimulating fibronectin synthesis, in particular a corn extract, such an extract notably being sold by the Applicant under the name Deliner™;
an agent for protecting the fibroblast growth factor (FGF2) of the extracellular matrix against its degradation and/or denaturing, notably an extract of Hibiscus abelmoscus as described in the patent application in the name of the Applicant filed under the number FR 0 654 316 and/or an agent for stimulating fibroblast growth, for example an extract of fermented soya containing peptides, known under the name Phytokine™ sold by the Applicant and also described in patent application EP 1 119 344 B1 (Laboratoires Expanscience), and preferentially a combination of these two extracts;
an agent for stimulating laminin synthesis, in particular an extract of biotechnologically modified malt, such an extract being notably sold by the Applicant under the name Basaline™; and an agent for stimulating lipid synthesis, for instance an extract of biotechnologically modified potato (Solanum tuberosum) sold by the Applicant under the name Lipidessence™;
an agent for stimulating the expression and/or activity of hyaluronane synthase 2 (HAS2) such as the plant extracts described in patent application FR2 893 252 A1 and in particular an aqueous extract of galanga (Alpinia galanga) sold under the name Hyalufix™ by the Applicant;
an agent for stimulating the synthesis of lysyl oxidase like (LOXL) such as those described in patent application FR 2 855 968, and in particular a dill extract sold under the name Lys'lastine™ by the Applicant;
one or more antipollution agents such as an extract of Argania spinosa leaves sold under the name Arganyl™ by the Applicant or an extract of Moringa oleifera seeds sold under the name Purisoft™ by the Applicant or an extract of Eperua falcata root sold under the name Eperuline™ by the Applicant;
an agent for stimulating intracellular ATP synthesis, notably an extract of the alga Laminaria digitata;
an agent with overall anti-ageing action, notably for combating pigmentation marks, in particular niacinamide or vitamin B3;
an antibacterial agent and/or sebum-regulating agent and/or sebum-absorbing agent such as retinoids, sarcosine, zinc salts, in particular zinc gluconate or zinc salicylate, azelaic acid, and/or derivatives and mixtures thereof, an extract of Orthosiphon stamineus sold under the name MAT XS™ Bright by the Applicant, an extract of Bixa orellana sold under the name Bix'Activ™ by the Applicant, the antibacterial extracts described in patent application FR 2 863 893, and in particular a boldo extract, notably the product sold under the name Betapur™ by the Applicant, or talc,
and any mixture thereof.

The use of the porous spheres according to the present invention is particularly advantageous insofar as it allows immediately effective and long-lasting action on any type of skin, including the scalp, on skin integuments, preferentially the hair, and on mucous membranes, in particular skin showing unaesthetic manifestations or signs of discomfort.

Preferentially, the porous spheres according to the invention, preferentially in the form of a cosmetic composition according to the invention, are applied to at least one area of the body where there are unaesthetic and/or uncomfortable manifestations, this or these areas preferentially being a surface of the body chosen from facial skin, including the forehead, the cheeks, the nose, the temples, the T area (forehead, nose and chin), under the eyes, the periorbital area, in particular the area under the eyes, the chin, the scalp, the neck, the back, the shoulders, the forearms, the thorax, the hands, the hair, the beard, the eyelashes, the eyebrows and/or the chest, in particular the legs, the feet, the armpits, the hands, the neck, the neckline, the tummy, the arms, the thighs, the hips, the buttocks, the waist, the torso, the back, the face and/or the scalp, the contour of the lips and the hair.

A subject of the present invention is thus also a cosmetic care process comprising the topical application to at least one area of skin and/or skin integuments, notably the hair, of the porous spheres according to the invention, preferentially to at least one surface chosen from a surface of the body chosen from facial skin, including the forehead, the cheeks, the nose, the temples, the T area (forehead, nose and chin), under the eyes, the periorbital area, in particular the area under the eyes, the chin, the scalp, the neck, the back, the shoulders, the forearms, the thorax, the hands, the hair, the beard, the eyelashes, the eyebrows and/or the chest, in particular the legs, the feet, the armpits, the hands, the neck, the neckline, the tummy, the arms, the thighs, the hips, the buttocks, the waist, the torso, the back, the face and/or the scalp, the contour of the lips and the hair.

The spheres according to the invention are particularly suitable for their uses on any type of skin, mucous membranes and/or integuments, notably Caucasian, Asiatic or African skin and also on any nature of skin, notably sensitive skin, skin with atopic tendency, greasy skin and sensitive skin. They are particularly suitable for masking the first unaesthetic manifestations of ageing, notably the first expression wrinkles and/or for mature skin, i.e. the skin of men and women at least 50 years old, in particular menopausal women.

The cosmetic compositions according to the invention are preferentially of no-rinse type.

Advantageously, the porous spheres according to the invention, preferentially in the form of a composition intended for topical application, preferentially a cosmetic composition according to the invention, are used in regular topical application and preferentially at least once a day, advantageously twice a day. Preferentially, the cosmetic composition is applied to the skin.

The spheres may be used in powder form, but also in the form of a cosmetic ingredient formulated in liquid form. For their formulation as a cosmetic ingredient, the spheres are then preferentially suspended in glycerol and/or in another solvent, which is notably polar, such as water, alcohols, notably propanediol, glycols, notably butylene glycol, propylene glycol, polyols or a mixture thereof, preferentially a water-glycol mixture, more preferentially containing a glycol chosen from butylene glycol, propylene glycol, caprylyl glycol, hexylene glycol and mixtures thereof. Particularly advantageously, the spheres according to the invention are suspended in an aqueous solution containing glycerol, hexylene glycol, caprylyl glycol or a mixture thereof.

Advantageously, a subject of the invention is also a cosmetic treatment method for improving the appearance of the skin and/or skin integuments, preferentially the hair, and/or for improving the comfort of an individual in need thereof/who so desires, comprising the following steps:
identifying on the individual an area of skin whose appearance and/or comfort is to be improved and/or which shows unaesthetic manifestations to be hidden, and
topical application to this area of skin of a cosmetic composition containing the spheres according to the invention in an amount that is effective for improving the appearance and/or comfort of this area of skin and/or of skin integuments, preferentially the hair, advantageously in a sphere content of between 1×10⁻³ and 100% by weight, advantageously ranging from 1×10⁻² to 95%, preferably ranging from 0.01% to 80%, more preferentially from 0.05% to 50%, preferentially from 0.1% to 30%, preferentially up to 20%, more preferentially up to 10% and more preferentially from 0.5% to 5% by weight relative to the total weight of the composition.

The spheres according to the invention may also be used for improving the comfort of the skin, mucous membranes and/or skin integuments presenting a pathology.

The skin, mucous membranes and/or skin integuments are preferentially impaired, in particular in the context of a pathology chosen from the group consisting of reactive, inflammatory or atopic skin, acne, psoriasis, herpes, boils, folliculitis, abscesses, impetigo, ecthyma, erysipelas, wounds and/or acne scars and/or any combination thereof.

In a preferential embodiment of the invention, the spheres according to the invention are in the form of a pharmaceutical composition also comprising a pharmaceutically acceptable excipient and are present in the pharmaceutical composition in a sphere content of between 1×10⁻³ and 100% by weight, and advantageously ranging from 1×10⁻² to 95%, preferably ranging from 0.01% to 80%, more preferentially from 0.05% to 50%, preferentially from 0.1% to 30%, preferentially up to 20%, more preferentially up to 10%, and more preferentially from 0.5% to 5% by weight relative to the total weight of the composition.

Other aims, characteristics and advantages of the invention will emerge clearly to a person skilled in the art on reading the explanatory description, which makes reference to examples that are given purely as illustrations and shall not in any way limit the scope of the invention.

The examples form an integral part of the present invention, and any characteristic appearing to be novel relative to any prior art whatsoever, from the description taken in its entirety, including the examples, forms an integral part of the invention in its function and in its general nature. Thus, each example has a general scope. Moreover, in the examples, all the percentages are given on a weight basis, unless otherwise indicated, the temperature is expressed in degrees Celsius, unless otherwise indicated, and the pressure is atmospheric pressure, unless otherwise indicated.

### EXAMPLES

### Example 1: Porous Silica Spheres

A copolymer of styrene and of acrylic acid is prepared as follows: an amount of 230 mL of deionized water is added to a three-necked reaction flask equipped with a thermometer, a condenser and a magnetic stirrer, and under a nitrogen atmosphere. The water is heated to 80° C. and an amount of 10 g of styrene is added with stirring, followed by an amount of 100 mg of acrylic acid dissolved in an amount of 10 mL of deionized water, by syringe. An amount of 100 mg of ammonium persulfate is dissolved in 10 mL of deionized water and added by syringe to the mixture with stirring. The mixture is left stirring for 24 hours at 80° C. The colloidal polymer dispersion is cooled to room temperature and is purified by centrifugation, thus forming the polystyrene nanospheres having a mean particle size of 250 nm. The colloidal aqueous dispersion of polystyrene is diluted to 1% by weight in deionized water and 1% by weight of silica nanoparticles is added. The mixture is sonicated to prevent agglomeration of the particles.

The continuous oily phase contains 0.1% by weight of polyethylene glycol/perfluoropolyether surfactant in a fluorinated oil.

The aqueous colloidal dispersion and the oil are each injected into a microfluidic device having a junction for obtaining a droplet size of 50 μm, via syringes connected to pumps. The assembly is left at equilibrium until the monodisperse droplets are produced. These droplets are then collected. The collected droplets are then dried in an oven at 45° C. for 4 hours to give monodisperse sphere models. These polymer model spheres are then calcined by placing them on a silicone plate and heating them from room temperature to 500° C. over a period of 3 hours, and then maintaining the temperature of 500° C. for 2 hours. The spheres are cooled to room temperature for 3 hours. The monodisperse spheres thus obtained have a mean diameter of 15 microns.

According to an alternative embodiment, Example 1 may be repeated, performing the drying step by microwave irradiation, by drying under vacuum and/or in the presence of a dehydrating agent.

### Example 2: Variation of the Colours of Spheres

An amount of 0.5 mg of porous spheres is placed in a 10 ml clear glass flask having a bottom area of 6 cm². The colour is observed with the naked eye. Two samples of porous spheres are prepared according to Example 1, in which the polymer/silica weight ratio is 1/1 and 3/1. The sample prepared with the 1/1 ratio is white—the sample prepared with the 3/1 ratio has a blue colour.

Another sample of porous silica spheres was prepared according to Example 1, in which the polystyrene nanospheres have a mean particle size of 360 nm and a polymer/silica weight ratio of 3/1. The sample obtained has a green colour.

### Example 3: Porous Silica-Titanium Spheres

A sample of porous spheres containing silica and titanium was prepared according to the process described in Example 1, in which the polymer/metal oxide weight ratio is 3/1. The silica/titanium weight ratio is 9/1.

### Example 4: Evaluation of the Colour

An amount of 10 g of spheres obtained according to Example 1, on the one hand, and according to Example 3, on the other hand, was evaluated by microscope, spread in powder form between slide and cover slip and illuminated in various directions. It was observed that the spheres reflect the light, appear bright under the microscope and have tints of various colours according to the size of the sphere and of the pores.

The same experiment was performed on a composition of carbomer hydrogel type containing 1% by weight of spheres relative to the total weight of the composition.

Similarly, a composition of pure oil type containing 1% by weight of spheres relative to the total weight of the composition was prepared and observed. Moreover, an oil-in-water emulsion containing 1% by weight of spheres relative to the total weight of the composition was evaluated.

Besides having very good compatibility with the aqueous and oily media, the spheres had a soft feel on topical application, in particular in the compositions containing oil (oil or emulsion).

Finally, it was observed by microscope that the spheres in the evaluated compositions reflect light in all directions. They make it possible to improve the appearance of the skin, notably by hiding the unaesthetic manifestations. This was confirmed in vitro on a biopsy model.

### Example 5: Evaluation of the Improvement of the Comfort by a Cosmetic Composition According to the Invention

The spheres obtained in Example 1 were incorporated into a simple hydrogel of carbomer type at 0.5% and 0.1% by weight relative to the total weight of the composition, with vigorous stirring. The spheres are inert, do not swell again, and are in suspension in the medium. Dispersion is better with vigorous stirring by preventing agglomerates.

Similarly, the spheres were suspended at 0.05% by weight in water and at 0.05% by weight in a plant oil, are inert and do not swell again.

Similarly, the spheres were incorporated into an emulsion of facial care type at 1% by weight and evaluated by individuals trained in sensory analysis: they applied to their forearm the emulsion containing the spheres according to a defined procedure. It emerges from this evaluation that the trained individuals reported that the emulsion containing the spheres according to the invention affords a richer, more oily application sensation, longer penetration of the cream into the skin was observed, and a velvety finish was observed. Finally, a very soft sensation on the skin was described, with a slightly satiny effect of the skin. The spheres according to the invention thus improved the comfort of the skin.

### Example 6: Cosmetic Compositions According to the Invention

The porous spheres used are those obtained in Example 1, 2 or 3 in powder form.

### Example 6a): Fluid Facial Emulsion

TABLE 1

| | | Amount |
|---|---|---|
| Phase | Name | (% by total weight) |
| A | Water | 77.05 |
| A | Glycerol | 2.00 |
| A | Preserving agent | 1.00 |
| B | Carbomer (Rheocare ™) | 0.20 |
| C | Lauryl glucoside, polyglyceryl-2, | 2.00 |
| | dipolyhydroxystearate, glycerol, | |
| | cetearyl alcohol (Eumulgin ™ VL 75) | |
| C | Cetearyl alcohol | 1.00 |
| C | Cocoyl glycerides | 3.00 |
| C | Ethylhexyl stearate | 3.00 |
| C | Dicaprylyl carbonate | 4.00 |
| D | Sodium polyacrylate, dicaprylyl | 1.00 |
| | carbonate, polyglyceryl-3 caprate | |
| | (Cosmedia ™ Ace), | |
| E | Colourant CI 17200 | 0.10 |
| E | Pigments: mica, titanium dioxide, | 2.00 |
| | silica (Flamenco ™ summit magenta | |
| | M80H) | |
| F | Spheres according to the invention | 3.00 |
| F | Fragrance | 0.65 |

The emulsion is prepared by the usual methods in the field well known to a person skilled in the art, by introducing phase B into phase A with stirring until fully dispersed. The mixture is heated to 75-80° C., as is phase C, separately. Phase C is then added to the mixture with stirring. The mixture is left to cool with gentle stirring to room temperature and the compounds of phases E and F are added, one after the other. The whole is homogenized for 2 minutes. The pH is adjusted to 5.2.

### Example 6b): Facial Cream

TABLE 2

| | | Amount |
|---|---|---|
| Phase | Name | (% by total weight) |
| A | Sucrose polystearate, cetyl palmitate | 3.00 |
| A | Pentaerythrityl distearate | 1.00 |
| A | Caprylic/capric triglyceryl | 3.00 |
| A | Cocoyl caprylate/caprate | 3.00 |
| A | Dicaprylyl carbonate | 3.00 |
| A | Sodium polyacrylate | 0.70 |
| B | Water | 80.90 |
| B | Glycerol | 2.00 |
| B | Sodium stearoyl glutamate | 0.50 |
| B | Preserving agent | qs |
| C | Spheres according to the invention | 0.5 |
| C | Water | 1.75 |
| D | Fragrance | qs |
| E | pH adjuster (citric acid) | qs |

The cream is prepared by the usual methods in the field well known to those skilled in the art, by mixing phases A and B preheated to 75° C., followed by adding phases C and D while mixing and while adjusting the composition with phase E to a pH of 6.2 and to a viscosity of 15 000 mPa·s (measured with a Brookfield instrument (RVT; 23° C., spindle TC; 20 rpm)).

### Example 6c): Shampoo

TABLE 3

| | | Amount |
|---|---|---|
| Phase | Name | (% by total weight) |
| A | Water | 60.3 |
| A | Xanthan gum | 1.2 |
| B | Decyl glucoside | 14 |
| B | Dicaprylyl ether, decyl | 5 |
| | glucoside, glyceryl oleate | |
| B | Sodium cocoyl glutamate | 12 |
| B | Cocoyl glucoside, glyceryl | 2 |
| | oleate | |
| B | Glycerol | 3 |
| B | Preserving agent | qs |
| C | pH adjuster (citric acid) | qs |
| D | Fragrance | 0.5 |
| D | Spheres according to the | 0.01-10 |
| | invention | |

The shampoo is prepared by the usual methods in the field well known to those skilled in the art, by mixing the four phases and by adjusting the composition to a pH of 5.2 and to a viscosity of 2200 mPa·s (measured with a Brookfield instrument (RVT; 23° C., spindle 5; 50 rpm)).

1.-18. (canceled)

19. A cosmetic method comprising applying porous spheres comprising a metal oxide to akin, skin integuments, and/or mucous membranes, thereby improving the appearance or comfort of the skin, skin integuments, or mucous membranes.

20. The cosmetic method according to claim 19, in which the spheres have at least one of a mean diameter ranging from 0.5 μm to 100 μm and a mean porosity ranging from 0.10 to 0.90, and a mean pore diameter ranging from 50 nm to 999 nm.

21. The cosmetic method according to claim 19, in which the spheres have a mean diameter ranging from 0.5 μm to 100 μm and a mean porosity ranging from 0.10 to 0.80 and a mean pore diameter ranging from 50 nm to 999 nm.

22. The cosmetic method according to claim 19, in which the spheres have a mean diameter ranging from 1 μm to 100 μm.

23. The cosmetic method according to claim 19, in which the spheres have a mean pore diameter ranging from 220 nm to 300 nm.

24. The cosmetic method according to claim 19, in which the metal oxide is the oxide silica, titania, alumina or zirconia, cerium oxide, iron oxide, zinc oxide, indium oxide, tin oxide, chromium oxide, mixed metal oxide and combinations thereof.

25. The cosmetic method according to claim 24, in which the metal oxide is a silica or titanium oxide or a combination thereof.

26. The cosmetic method according to claim 19, in which the porous spheres comprise from 60% by weight to 99.9% by weight of metal oxide and from 0.1% by weight to 40% by weight of light-absorbing agents, relative to the total weight of the spheres.

27. The cosmetic method according to claim 19, in which the spheres have a mean diameter ranging from 1 μm to 75 μm and a mean porosity ranging from 0.45 to 0.65.

28. The cosmetic method according to claim 19, in which the spheres have a mean diameter ranging from 4.5 μm to 9.9 μm, a mean porosity ranging from 0.45 to 0.65 and a mean pore diameter ranging from 220 nm to 300 nm.

29. The cosmetic method according to claim 19, for reducing the visibility of the unaesthetic manifestations of the skin, skin integuments or mucous membranes, skin relief or texture irregularities, and skin colour irregularities.

30. The cosmetic method according to claim 19, in which the spheres are in a cosmetic composition, in a concentration ranging from 1×10⁻³ to 100% by weight relative to the total weight of the composition.

31. The cosmetic method according to claim 30, for improving the organoleptic properties of the cosmetic composition.

32. A cosmetic composition, characterized in that it contains porous spheres as described in claim 19.

33. The cosmetic composition according to claim 32, characterized in that it is in the form of a serum, a lotion, a cream, a shampoo, a hair conditioner, an oil, a milk, an ointment, a paste, a foam, an emulsion, a hydrogel, a shower gel, a mask, a lacquer, a spray, a wax, a powder, a makeup powder, or a stick.

34. The cosmetic composition according to claim 32, characterized in that it is in the form of a slightly gelled composition or comprises an oily phase, and is of oily composition type or oil-in-water or water-in-oil emulsion or lotion type.

35. A dermatological composition, characterized in that it contains porous spheres as described in claim 19.

36. A dermatological composition, according to claim 35, for improving the comfort of skins, skin integuments or mucous membranes presenting a pathology.

37. The cosmetic method according to claim 19, in which the spheres have a mean diameter ranging from 4.5 μm to 9.9 μm.

38. The cosmetic method according to claim 19, in which the spheres are in a cosmetic composition, in a concentration ranging from 0.5% to 5% by weight, relative to the total weight of the composition.
